(19) United States
(12) Patent Application Publication
(10) Pub. No.: US 2007/0112363 A1
(43) Pub. Date: May 17, 2007

(54) Apparatus including multiple invaginators for restoring a gastroesophageal flap valve and method

(75) Inventor: John M. Adams, Sammamish, WA (US)

Correspondence Address: GRAYBEAL JACKSON HALEY LLP, Suite 350, 155-108th Avenue N.E., Bellevue, WA, 11573, US

(73) Assignee: EndoGastric Solutions, Inc.

(21) Appl. No.: 11/274,045

(22) Filed: Nov. 15, 2005

(51) Int. Cl. A61B 17/08

(52) U.S. Cl. 606/153

(57) ABSTRACT

A transoral gastroesophageal flap valve restoration assembly comprises a pair of tissue grippers. The device comprises an elongated member having a distal end arranged for being fed down an esophagus in communication with a stomach and a tissue shaper carried on the distal end of the longitudinal member. The tissue shaper comprises a first member adjacent the distal end of the elongated member and a second member. The first and second members are hingedly coupled to receive the stomach tissue to be shaped there between. The distal end of the elongated member has one of the tissue grippers that grips esophageal tissue oral of a Z line and the first member of the tissue shaper has the other tissue gripper that grips stomach tissue aboral of the Z line. A tissue fastener maintains the Shaped stomach tissue.

### FIELD OF THE INVENTION

The present invention generally relates to a device for the restoration of a gastroesophageal flap valve (GEFV) as a therapy for gastroesophageal reflux disease (GERD). The present invention more particularly relates to a transoral endoscopic device for such use having multiple invaginators that enable valve restoration for a wide patient population including those with a hiatal hernia and without invasive gripping of esophageal or stomach tissue.

### BACKGROUND

A hiatal hernia is an anatomical abnormality in which part of the stomach protrudes through the diaphragm and up into the chest. Hiatal hernias are present in approximately 15% of the population and its occurrence increases with age. Recent studies estimate that it is present in 60% of those over 60 years of age.

Normally, the esophagus or food tube passes down through the chest, crosses the diaphragm, and enters the abdomen through a hole in the diaphragm called the esophageal hiatus. This “hole” is a muscular tube or channel of about two to three vertebrae in length. Just below the diaphragm, the esophagus joins the stomach at the gastroesophageal junction. In individuals with hiatal hernias, the opening of the esophageal hiatus (hiatal opening) is larger than normal, and a portion of the upper stomach slips up or passes (herniates) through the hiatus and into the chest. Although hiatal hernias are occasionally seen in infants where they probably have been present from birth, most hiatal hernias in adults are believed to have developed over many years

It is thought that hiatal hernias develop as a part of permanent positive pressure in the abdomen and negative pressure in the chest with thousands of daily unsynchronized movements of the esophagus and diaphragm. Over time, the phrenoesophageal bundles elongate, allowing the gastroesophageal junction (GEJ) to slip into the chest. Widening is currently thought of as being the result of mechanical dilatation and recurrent inflammation in the herniated stomach (often referred to as the acid chamber) which leads to periesophagitis and retraction of the diaphragmatic muscle over time. As a result of the large opening, part of the stomach “slips” into the chest. Another potentially contributing factor is include an abnormally loose attachment of the esophagus to the diaphragm which allows the esophagus and stomach to slip upwards.

Hiatal hernias are categorized as being either sliding or para-esophageal. Sliding hiatal hernias are those in which the junction of the esophagus and stomach, referred to as the gastro-esophageal junction, and part of the stomach protrude into the chest. The junction may reside permanently in the chest, but often it juts into the chest only during a swallow. This occurs because with each swallow the muscle of the esophagus contracts causing the esophagus to shorten and to pull up the stomach. When the swallow is finished, the herniated part of the stomach falls back into the abdomen. Para-esophageal hernias are hernias in which the gastro-esophageal junction stays where it belongs (attached at the level of the diaphragm), but part of the stomach passes or bulges into the chest beside the esophagus. The para-esophageal hernias themselves remain in the chest at all times and are not affected by swallows.

A para-esophageal hiatal hernia that is large, particularly if it compresses the adjacent esophagus, may impede the passage of food into the stomach and cause food to stick in the esophagus after it is swallowed. Ulcers also may form in the herniated stomach due to the trauma caused by food that is stuck or acid from the stomach. Fortunately, large para-esophageal hernias are uncommon.

The vast majority of hiatal hernias are of the sliding type. The larger the hernia, the more likely it is to cause symptoms. When hiatal hernias produce symptoms, they may also be associated with gastro-esophageal reflux disease (GERD), to be described herein after, or its complications. GERD can occur because the formation of the hernia often interferes with the natural barrier which prevents acid from refluxing from the stomach into the esophagus. Patients with GERD are much more likely to have a hiatal hernia than individuals not afflicted by GERD. Thus, it is clear that hiatal hernias contribute to GERD.

Normally, there are several mechanisms to prevent acid from flowing backwards (refluxing) up into the esophagus. One mechanism involves a band of esophageal muscle where the esophagus joins the stomach called the lower esophageal sphincter that remains contracted most of the time to prevent acid from refluxing or regurgitating. The sphincter only relaxes when food is swallowed so that the food can pass from the esophagus and into the stomach. The sphincter normally is attached firmly to the diaphragm in the hiatus, and the muscle of the diaphragm wraps around the sphincter. The muscle that wraps around the diaphragm augments the pressure of the contracted sphincter to further prevent reflux of acid.

Another mechanism that prevents reflux is the valve-like tissue at the junction of the esophagus and stomach just below the sphincter. The esophagus normally enters the stomach tangentially so that there is a sharp angle between the esophagus and stomach. The thin piece of tissue in this angle, composed of esophageal and stomach wall, forms a valve that can close off the opening to the esophagus when pressure increases in the stomach, for example, during a belch.

When a hiatal hernia is present, two changes occur. First, the sphincter slides up into the chest while the diaphragm remains stationery. As a result, the pressure normally generated by the diaphragm overlying the sphincter and the pressure generated by the sphincter no longer overlap, and as a result, the total pressure at the gastro-esophageal junction decreases. Second, when the gastro-esophageal junction and stomach are pulled up into the chest with each swallow, the sharp angle where the esophagus joins the stomach becomes less sharp and the valve-like effect is lost. Both changes promote reflux of acid

Hiatal hernias are diagnosed incidentally when an upper gastrointestinal x-ray or endoscopy is done during testing to determine the cause of upper gastrointestinal symptoms such as upper abdominal pain. On both the x-ray and endoscopy, the hiatal hernia appears as a separate “sac” lying between what is clearly the esophagus and what is clearly the stomach. This sac is delineated by the lower esophageal sphincter above and the diaphragm below.

Treatment of large para-esophageal hernias causing symptoms requires surgery. During surgery, the stomach is accessed invasively through incisions made in the abdomen. The stomach is pulled down into the abdomen, the esophageal hiatus is made smaller, and the esophagus is attached to the diaphragm with sutures. Although the procedure restores the normal anatomy, it is invasive, requiring weeks or even months of recuperation before all normal activity may be resumed.

As will be seen subsequently, the present invention provides an alternative procedure for treating hiatal hernias. Instead of being surgically invasive, the new procedure, according to the various embodiments described herein after, may be performed transorally without the need for invasive incisions. As a result, patients are able to recover much more quickly and return to normal activity within a few days.

Gastroesophageal reflux disease (GERD) is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV gastroesophageal flap. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laparoscopic surgical procedures are available for treating GERD. One surgical approach is the Nissen fundoplication. The Nissen approach typically involves a 360-degree wrap of the fundus around the gastroesophageal junction. The procedure has a high incidence of postoperative complications. The Nissen approach creates a 360-degree moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction. Another surgical approach is the Hill repair. In the Hill repair, the gastroesophageal junction is anchored to the posterior abdominal areas, and a 180-degree valve is created by a system of sutures. The Hill procedure restores the moveable flap, the cardiac notch and the Angle of His. However, all of these surgical procedures are very invasive, regardless of whether done as a laparoscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak, in part due to the fact, that the esophagus is not covered by serosa, a layer of very sturdy, yet very thin tissue, covering and stabilizing all intraabdominal organs, similar like a fascia covering and stabilizing muscle. Involvement of esophageal tissue in the repair of a gastroesophageal flap valve poses unnecessary risks to the patient, such as an increased risk of fistulas between the esophagus and the stomach.

A new and improved apparatus and method for restoration of a gastroesophageal flap valve is fully disclosed in U.S. Pat. No. 6,790,214, issued Sep. 14, 2004, is assigned to the assignee of this invention, and is incorporated herein by reference. That apparatus and method provides a transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

GEFV restoration and treatment for related hiatal hernias in a manner to avoid invasive surgery has been proposed. For example, as described in copending U.S. application Ser. No. 11/203,680 filed Aug. 12, 2005 and which is incorporated herein by reference, a method of treating a stomach disorder comprises providing a transoral gastroesophageal valve restoration device, feeding the device down the esophagus into the stomach, forming a gastroesophageal valve with the device from within the stomach, fastening stomach tissue to maintain the gastroesophageal valve, and securing the stomach to the diaphragm from within the stomach.

As further described, the step of securing the stomach to the diaphragm may include fastening the stomach to a crus of the diaphragm, such as the right crus. The described method further contemplates gripping the esophagus and displacing the esophagus until the stomach is completing within the diaphragm before securing the stomach to the diaphragm. The steps of gripping the esophagus and displacing the esophagus until the stomach is completely within the diaphragm is preferably performed before the step of forming the gastroesophageal valve with the device from within the stomach. The present invention provides alternative approaches to treating a hiatal hernia and/or restoring a GEFV.

### SUMMARY

The invention provides a transoral gastroesophageal flap valve restoration assembly comprising an elongated member having a distal end arranged for being fed down an esophagus in communication with a stomach, a tissue shaper carried on the distal end of the longitudinal member, the tissue shaper comprising a first member adjacent the distal end of the elongated member and a second member. The first and second members are hingedly coupled to receive the stomach tissue to be shaped there between and the distal end of the elongated member has a tissue gripper that grips esophageal tissue oral of a Z line. The first member of the tissue shaper has a tissue gripper that grips stomach tissue aboral of the Z line. The device further comprises a tissue fastener that maintains the shaped stomach tissue.

The assembly may further comprise a window between the tissue gripper of the elongated member and the tissue gripper of the first member of the tissue shaper to permit visualization of the Z line. The tissue fastener may be aboral of the window.

At least one of the tissue gripper of the elongated member and the tissue gripper of the first member of the tissue shaper may be a noninvasive gripper. Alternatively, both the tissue gripper of the elongated member and the tissue gripper of the first member of the tissue shaper are noninvasive grippers.

At least one of the tissue gripper of the elongated member and the tissue gripper of the first member of the tissue shaper may be a vacuum gripper. Both the tissue gripper of the elongated member and the tissue gripper of the first member of the tissue shaper may be vacuum grippers.

The elongated member may be arranged to axially translate after the gripper of the elongated member grips esophageal tissue and the gripper of the first member grips stomach tissue to cause the Z line to be aboral of an associated diaphragm.

The invention further provides a transoral gastroesophageal flap valve restoration assembly comprising an elongated member having a distal end arranged for being fed down an esophagus in communication with a stomach, the distal end of the elongated member having a first tissue gripper that grips esophageal tissue oral of a Z line and a second tissue gripper that grips stomach tissue aboral of the Z line The assembly further comprises a tissue shaper carried on the distal end of and hingedly coupled to the elongated member to cause stomach tissue to be shaped to be received between the elongated member and the tissue shaper when the tissue shaper hingedly pivots into engagement with the elongated member and a tissue fastener that maintains the shaped stomach tissue.

The invention still further comprises a transoral gastroesophageal flap valve restoration assembly comprising an elongated member having a distal end arranged for being fed down an esophagus in communication with a stomach, the distal end of the elongated member having a plurality of tissue grippers that together grip both esophageal tissue oral of a Z line and stomach tissue aboral of the Z line. The assembly further comprises a tissue shaper carried on the distal end of and hingedly coupled to the elongated member to cause stomach tissue to be shaped to be received between the elongated member and the tissue shaper when the tissue shaper hingedly pivots into engagement with the elongated member, and a tissue fastener that maintains the shaped stomach tissue.

The invention further provides a method of restoring a gastroesophageal flap valve associated with an esophagus, a stomach and a Z line that transitions esophageal tissue and stomach tissue. The method comprises the steps of concurrently gripping esophageal tissue oral of the Z line and stomach tissue aboral of the Z line, axially translating the gripped tissue until the Z line is within an associated diaphragm, and

manipulating stomach tissue from within the stomach to restore the gastroesophageal flap valve.

The concurrently gripping step may include substantially simultaneously gripping the esophageal tissue oral of the Z line and stomach tissue aboral of the Z line.

The concurrently gripping step may include non-invasively gripping the esophageal tissue oral of the Z line and stomach tissue aboral of the Z line.

The concurrently gripping step may include vacuum gripping the esophageal tissue oral of the Z line and stomach tissue aboral of the Z line.

The manipulating step may include folding stomach tissue into a gastroesophageal flap valve shape. The method may further comprise the step of fastening the manipulated tissue to maintain the restored gastroesophageal flap valve.

The method may further comprise first gripping the esophagus oral of the Z line, translating the esophagus in an oral direction, and while maintaining a grip on the esophagus oral of the Z line, gripping the stomach tissue aboral of the Z line to concurrently grip esophageal tissue oral of the Z line and stomach tissue aboral of the Z line. The gripping steps may include non-invasively gripping the esophageal tissue oral of the Z line and stomach tissue aboral of the Z line. The gripping steps may include vacuum gripping the esophageal tissue oral of the Z line and stomach tissue aboral of the Z line. The manipulating step may include folding stomach tissue into a gastroesophageal flap valve shape. The method may further comprise the step of fastening the manipulated tissue to maintain the restored gastroesophageal flap valve.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a front cross-sectional view of the esophageal-gastro-intestinal tract from a lower portion of the esophagus to the duodenum;

FIG. 2 is a partial perspective view with portions cut away of a stomach, esophagus, and diaphragm illustrating a hiatal hernia which may be treated according to an embodiment of the invention;

FIG. 3 is a side view of an apparatus according to an embodiment of the invention placed in an initial position within an esophagus;

FIG. 4 is a side view of the apparatus of FIG. 3 concurrently gripping esophageal and stomach tissue according to an embodiment of the invention;

FIG. 5 is a side view of the apparatus of FIG. 3 after translating the esophageal and stomach tissue aborally according to an embodiment of the invention;

FIG. 6 is a side view of the apparatus of FIG. 3 showing the initial folding of stomach tissue to restore a GEFV according to an embodiment of the invention;

FIG. 7 is a side view of the apparatus of FIG. 3 showing the stomach tissue further folded according to an embodiment of the invention;

FIG. 8 is a side view of the apparatus of FIG. 3 showing the stomach tissue fully folded according to an embodiment of the invention;

FIG. 9 is a side view of the apparatus of FIG. 3 showing the stomach tissue fully folded and fastened according to an embodiment of the invention;

FIG. 10 is a side view showing a restored GEFV according to an embodiment of the invention;

FIG. 11 is a side view of an apparatus according to another embodiment of the invention placed in an initial position within an esophagus and stomach;

FIG. 12 is a side view of the apparatus of FIG. 11 according to another embodiment of the invention gripping esophageal tissue oral of the Z;

FIG. 13 is a side view of the apparatus of FIG. 11 according to another embodiment of the invention after pulling the gripped esophageal tissue orally; and

FIG. 14 is a side view of the apparatus of FIG. 11 concurrently gripping esophageal and stomach tissue before initial folding of the stomach tissue to restore the GEFV according the another embodiment of the invention.

### DETAILED DESCRIPTION

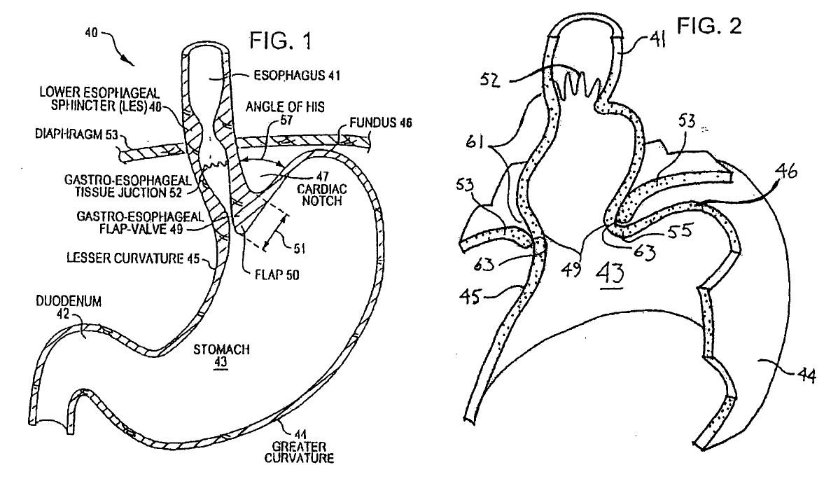

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue.

The fundus 46 of the greater curvature 44 forms the superior portion of the stomach 43, and traps gas and air bubbles for burping. The esophageal tract 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion.

The moveable portion of the GEFV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43. It is about 4 to 5 cm long (51) at it longest portion, and its length may taper at its anterior and posterior ends.

The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES) in the neck near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

FIG. 2 is a perspective view, with portions cut away, of stomach 43, esophagus 41, diaphragm 53, and hiatal hernia 61 which may be treated according to an embodiment of the present invention. As previously mentioned, a principal reason for regurgitation associated with GERD is the mechanical failure of the deteriorated (or reflux appearance) gastroesophageal flap of the GEFV to close and seal against the higher pressure in the stomach. Due to reasons including lifestyle, a Grade I normal gastroesophageal flap of the GEFV may deteriorate into a Grade III deteriorated gastroesophageal flap. The anatomical results of the deterioration include moving a portion of the esophagus 41 that includes the gastroesophageal junction 52 and LES (not shown) toward the mouth through the hiatus 63 into the chest to create the hiatal hernia 61. This greatly reshapes the anatomy aboral of the gastroesophageal junction 52 and forms a flattened fundus 46.

Dr. Hill and colleagues developed a grading system to describe the appearance of the GEFV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEFV 49 illustrated in FIG. 1 is a Grade I flap valve that is the least likely to experience reflux. The deteriorated gastroesophageal flap 55 of the GEFV 49 illustrated in FIG. 2 is a Grade IV flap valve. The Grade IV flap valve is the most likely to experience reflux. Grades II and III reflect intermediate grades of deterioration and, as in the case of III, a high likelihood of experiencing reflux. With the deteriorated GEFV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently is a device, assembly, and method which may be employed to advantage according to an embodiment of the invention to treat the hiatal hernia 61 and restore the normal gastroesophageal flap valve anatomy.

Referring now to FIG. 3, it shows a device 100 according to an embodiment of the present invention. The device 100 includes a longitudinal member 102 for transoral placement of the distal end of the device 100 into the stomach. The device further includes a first member 104, hereinafter referred to as the chassis, and a second member 106, hereinafter referred to as the bail. The chassis 104 and bail are hingedly coupled at 107. The chassis 104 and bail 106 form a tissue shaper which, as described subsequently in accordance with this embodiment of the present invention, shapes tissue of the stomach into the flap of a restored gastroesophageal flap valve. The chassis 104 and bail 106 are carried at the distal end of the longitudinal member 102 for placement in the stomach.

The device 100 has a longitudinal passage 101 to permit an endoscope 110 to be guided through the device and into the stomach. This permits the endoscope to serve as a guide for guiding the device 100 through the patient's throat, down the esophagus, and into the stomach. It also permits the gastroesophageal flap valve restoration procedure to be viewed at each stage of the procedure.

As will be seen subsequently, to facilitate shaping of the stomach tissue, the stomach tissue is drawn in between the chassis 104 and the bail 106. In use, the device 100 is fed down the esophagus 41 with the bail 106 substantially in line with the chassis 104 as shown in FIG. 3. To negotiate the bend of the throat, and as described in the aforementioned referenced application, the chassis 104 and bail 106 are rendered flexible. The chassis 104 may be rendered flexible by being formed of flexible material and/or by incorporating slots (not shown). The bail 106 is preferably rendered flexible by including hingedly coupled links 112. Further details concerning the flexibility of the chassis 104 and the bail 106 may be found in copending application Ser. No. 11/001,666, filed Nov. 30, 2004, entitled FLEXIBLE TRANSORAL ENDOSCOPIC GASTROESOPHAGEAL FLAP VALVE RESTORATION DEVICE AND METHOD, which application is incorporated herein by reference.

As further shown in FIG. 3, the device 100 further includes at least two tissue grippers 114 and 116. The tissue grippers 114 and 116 are non-invasive tissue grippers. The first tissue gripper 114 is in the form of an invaginator having vacuum orifices 115 that vacuum grip the tissue. The first gripper 114 is carried by the elongated member 102. The second tissue gripper 116 is also in the form of an invaginator having vacuum orifices 117 that also vacuum grip the tissue. The second gripper 116 is carried by the chassis 104. However, the second gripper 116 may alternatively be thought of as being carried by the elongated member 102 if the chassis 104 is considered an extension of the elongated member 102.

With continued reference to FIG. 3, the device 100 further comprises a fastener deployer 140. The fastener deployer includes at least one fastener deployment guide 142. The fastener deployment guide 142 takes the form of a guide lumen. Although only one guide lumen 142 is shown, it will be appreciated that the device 100 may include a plurality of such lumens without departing from the invention. The guide lumen terminates at a delivery point 144 where a fastener is driven from the device 100 and into, for example, the molded stomach tissue. The fastener deployer may also be used, according to an embodiment, to secure the stomach to the diaphragm.

The device 100 further includes a window 130 within the chassis 104. The window is formed of a transparent or semi-transparent material and is located between the grippers 114 and 116. The window 130 permits gastroesophageal anatomy, and more importantly the gastroesophageal junction (Z-line) to be viewed with the endoscope 110. The window includes a location marker 132 which has a known position relative to the fastener delivery point 144. Hence, by aligning the marker with a known anatomical structure, the fastener will be delivered a known distance from or at a location having a predetermined relation to the marker. For example, by aligning the marker with the Z-line, it will be know that the fastener will be placed aboral of the Z-line and that serosa tissue will be fastened to serosa tissue. As previously mentioned, this has many attendant benefits.

The device 100, as shown in FIG. 3, is placed so that the first gripper 114 may grip esophageal tissue oral of the Z line 52 and the second gripper may grip tissue of a hiatal hernia aboral of the Z line. This is made possible by visualizing the Z line with the endoscope through the window 130. The vacuum gripping of the esophagus 41 and hiatal hernia 61 may be used to particular advantage in the treatment of the hiatal hernia 61. As will be seen subsequently, upon being thus gripped, the device is moved aborally toward and into the stomach to pull the gripped tissue into the stomach and also within the diaphragm to eliminate the hiatal hernia.

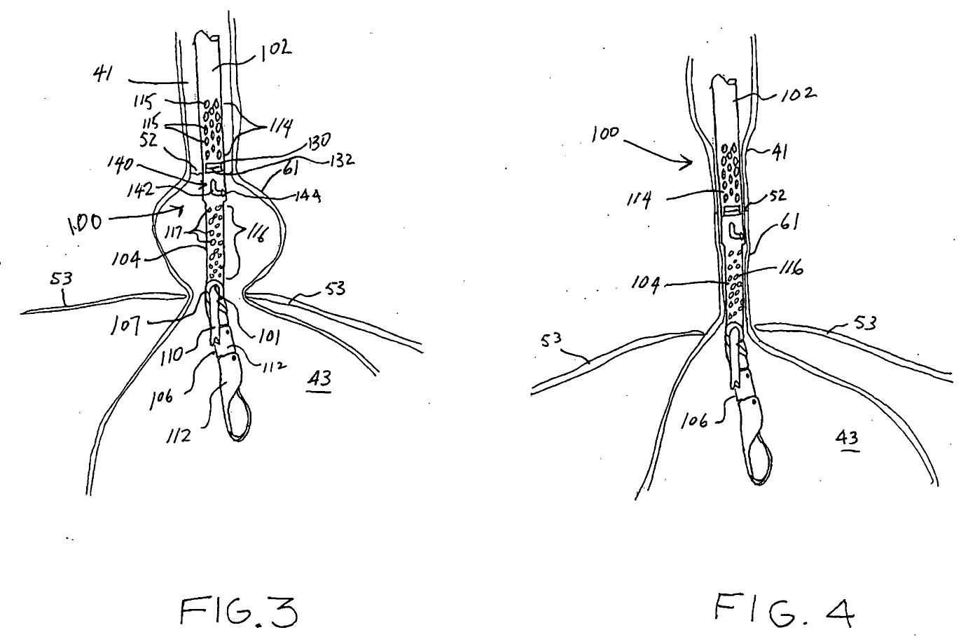

Referring now to FIG. 4, it shows the device 100 with the vacuum grippers 114 and 116 concurrently activated. The gripper 116 is gripping the herniated tissue 61 and the gripper 114 is gripping esophageal tissue oral of the Z line 52. It may be noted that the window 130 is aligned with the Z line 52. At this point the device 100 has not been moved towards the stomach.

To assist the second gripper 116 in gripping the herniated tissue 61, it may be desirable to first activate the first gripper 114. This will serve to block the air passageway of the esophagus 41. Then, with the air passageway of the esophagus blocked, the second gripper 116 may be activated.

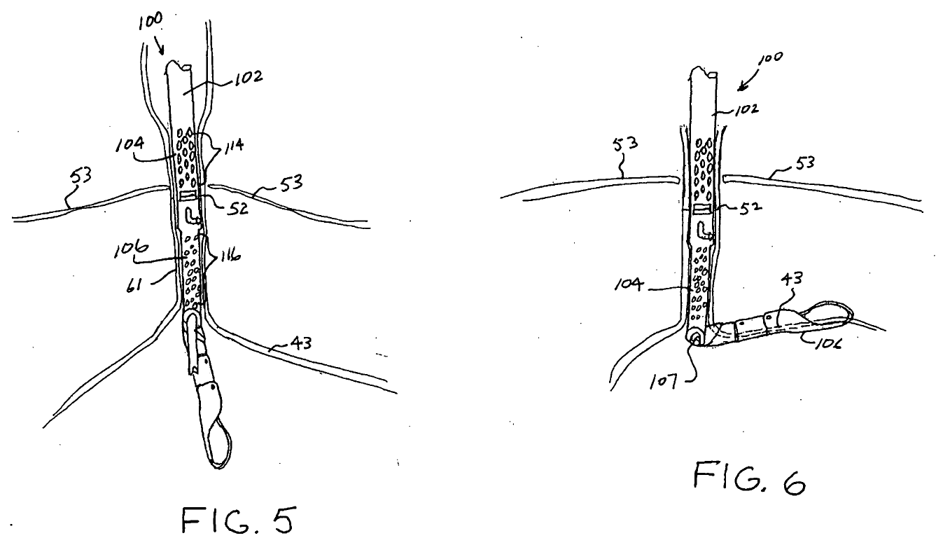

Once the first gripper 114 and second gripper 116 are concurrently activated, the device 100 is ready to be translated axially towards the stomach. Preferably, the device is moved orally until the Z line 52. The device will then appear as shown in FIG. 5. Here may it may be noted that the herniated tissue 61 is still held by the first gripper 61 and is now within the diaphragm 53. With the herniated tissue 61 within the diaphragm 53, and the Z line within the diaphragm 53, The stomach tissue 43 may now be folded to restore the GEFV.

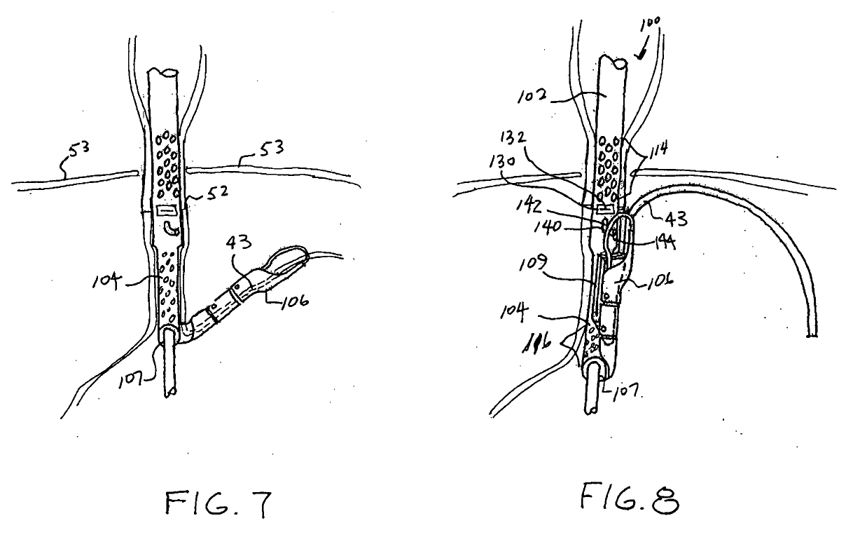

FIG. 6 illustrates this next step. Here it may be seen that the bail 106 is beginning to be pivoted about pivot point 107. As it rotates, it also engages and takes the stomach tissue 43 along with it. At this time, both the first and second grippers 114 and 116 respectively are active and stabilizing the esophagus and stomach tissue just aboral of the Z line. FIG. 7 shows this process continuing.

Referring now to FIG. 8, it may now be seen that the chassis 104 and bail 106 have been brought together. Throughout the pivoting of the bail 106, the first and second grippers 114 and 116 respectively have remained steadfast in gripping and stabilizing the esophageal and stomach tissue. With the bail 106 now meeting the chassis 104, The stomach tissue is folded into a fold 109 of a flap of stomach tissue. The folded tissue is now ready to receive at least one fastener to maintain the flap of tissue.

Here it will be noted that the stomach tissue aboral of the Z-line 52 is confined between the bail 106 and chassis 104 to create the fold 109. The fold is also adjacent the fastener delivery point 144 at the end of the fastener guide lumen 142. Since the fastener deployment point 144 is a known predetermined distance from the marker 132 of the window 130, and since the marker 132 is aligned with the Z-line 52, when a fastener is delivered from the fastener deployer of the device, the fastener will exit the fastener delivery point 144 at a point known to be aboral of the Z-line 52. This assures that only serosa tissue is being adhered to serosa tissue in the fixation of the stomach tissue in creating the flap 109 of stomach tissue.

With the tissue layers forming fold 109 now disposed within the mold of the chassis 104 and bail 106, the bail 106 may now be locked with respect to the chassis 104. It is now time to fasten the tissue layers together by ejecting a fastener from the fastener deployer lumen 142 at the fastener delivery point 144.

Before a fastener is ejected from the fastener deployer lumen 142, the stomach may be inflated through the endoscope 110. The stomach may be inflated to a point where one has a good view of the tissue fold and bail 106.

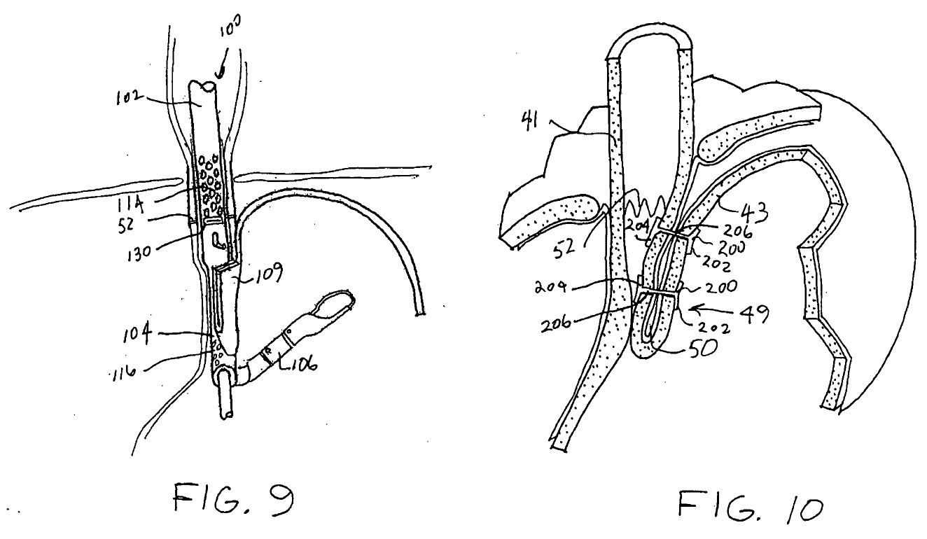

FIG. 9 shows the assembly after the tissue fold 109 is fastened. The bail 104 is retracted from the chassis 104 to expose the fold 109 of stomach tissue. The grippers may now be deactivated and the device may be rotated for forming a further fold if necessary. After the device is rotated, the Z line may be visualized through the window 130 to make sure that the device is properly aligned with the Z line 52. The grippers may 114 and 116 may once again be activated and another fold produced as described herein.

Once the foregoing procedure is completed, a flap 50 of a GEFV 49 is produced to restore the GEFV and provide GEFV functionality. This may be seen in FIG. 10. Here the fasteners 200 may be observed. The fasteners may generally include a first member 202 and second member 204 that are substantially parallel to each other and substantially perpendicular to a connecting member 206 that connects the first member 202 to the second member 204. The tissue is thus held between the first member 202 and the second member 204. For a complete description of such fasteners and the manner in which they may be deployed, reference may be had to co-pending application Ser. No. 11/043,903, filed Jan. 25, 2005, entitled SLITTED TISSUE FIXATION DEVICE AND ASSEMBLIES FOR DEPLOYING SAME, which application is incorporated herein by reference.

As may be noted in FIG. 10, the hiatal hernia no longer exists. Further, The tissue forming the fold 50 is aboral of the Z line 52. This results in good fixation and proper GEFV function.

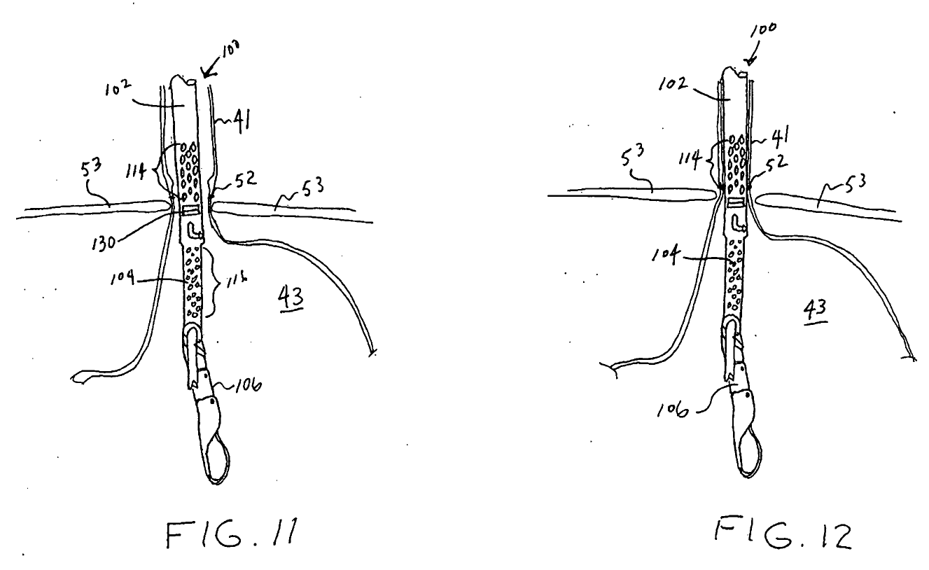

FIGS. 11-14 show another condition that may be addressed by the device 100. Here, it will be noted that there is a deteriorated GEFV without a hiatal hernia. The GEFV may be restored by the device 100 as described below.

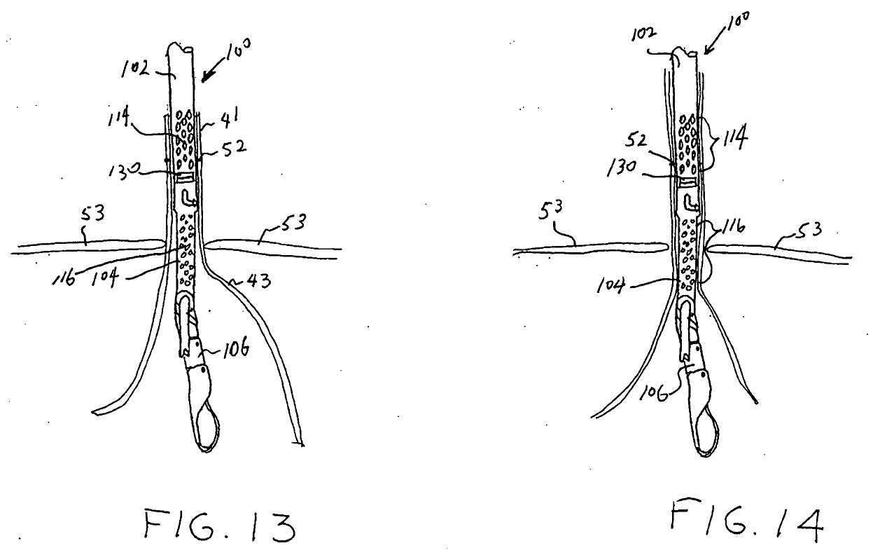

FIG. 11 shows the device 100 placed in an initial position. The window 130 is aligned with the diaphragm 53. With the device thus positioned, the first gripper 114 is activated to grip the esophagus 41. This is shown in FIG. 12. Next, with the esophagus firmly gripped by the first gripper 114, the device is drawn upward in an oral direction as shown in FIG. 13. This causes the stomach tissue just aboral of the Z line 52 to be drawn into close proximity with the second gripper 116. The second gripper may now be activated as shown in FIG. 14. Now the stomach tissue just aboral of the Z line is firmly held by the second gripper 116. Now, with the first gripper 114 and second gripper 116 concurrently gripping esophageal tissue and tissue aboral of the Z line, the device may be translated aborally towards the stomach until it reaches the position shown in FIG. 5. The procedure as previously described and as shown in FIGS. 6-9 may then be employed to restore the GEFV.

While particular embodiments of the present invention have been shown and described, modifications may be made, and it is thereto intended in the appended claims to cover all such changes and modifications which fall within the true spirit and scope of the invention.

What is claimed is:

1. A transoral gastroesophageal flap valve restoration assembly comprising:
an elongated member having a distal end arranged for being fed down an esophagus in communication with a stomach;
a tissue shaper carried on the distal end of the longitudinal member, the tissue shaper comprising a first member adjacent the distal end of the elongated member and a second member, the first and second members being hingedly coupled to receive the stomach tissue to be shaped there between, the distal end of the elongated member having a tissue gripper that grips esophageal tissue oral of a Z line and the first member of the tissue shaper having a tissue gripper that grips stomach tissue aboral of the Z line; and
a tissue fastener that maintains the shaped stomach tissue.

2. The assembly of claim 1, further comprising a window between the tissue gripper of the elongated member and the tissue gripper of the first member of the tissue shaper to permit visualization of the Z line.

3. The assembly of claim 2 wherein the tissue fastener is aboral of the window.

4. The assembly of claim 1, wherein at least one of the tissue gripper of the elongated member and the tissue gripper of the first member of the tissue shaper is a noninvasive gripper.

5. The assembly of claim 4, wherein both the tissue gripper of the elongated member and the tissue gripper of the first member of the tissue shaper are noninvasive grippers.

6. The assembly of claim 1, wherein at least one of the tissue gripper of the elongated member and the tissue gripper of the first member of the tissue shaper is a vacuum gripper.

7. The assembly of claim 6, wherein both the tissue gripper of the elongated member and the tissue gripper of the first member of the tissue shaper are vacuum grippers.

8. The assembly of claim 1, wherein the elongated member is arranged to axially translate after the gripper of the elongated member grips esophageal tissue and the gripper of the first member grips stomach tissue to cause the Z line to be aboral of an associated diaphragm.

9. A transoral gastroesophageal flap valve restoration assembly comprising:
an elongated member having a distal end arranged for being fed down an esophagus in communication with a stomach, the distal end of the elongated member having a first tissue gripper that grips esophageal tissue oral of a Z line and a second tissue gripper that grips stomach tissue aboral of the Z line;
a tissue shaper carried on the distal end of and hingedly coupled to the elongated member to cause stomach tissue to be shaped to be received between the elongated member and the tissue shaper when the tissue shaper hingedly pivots into engagement with the elongated member; and
a tissue fastener that maintains the shaped stomach tissue.

10. The assembly of claim 1, further comprising a window between the first tissue gripper of the elongated member and the second tissue gripper of the elongated member to permit visualization of the Z line.

11. The assembly of claim 10 wherein the tissue fastener is aboral of the window.

12. The assembly of claim 9, wherein at least one of the tissue grippers of the elongated member is a noninvasive gripper.

13. The assembly of claim 12, wherein both of the tissue grippers of the elongated member are noninvasive grippers.

14. The assembly of claim 9, wherein at least one of the tissue grippers of the elongated member is a vacuum gripper.

15. The assembly of claim 14, wherein both the tissue grippers of the elongated member are vacuum grippers.

16. The assembly of claim 9, wherein the elongated member is arranged to axially translate after the first gripper of the elongated member grips esophageal tissue and the second gripper of the elongated member grips stomach tissue to cause the Z line to be aboral of an associated diaphragm.

17. A transoral gastroesophageal flap valve restoration assembly comprising:
an elongated member having a distal end arranged for being fed down an esophagus in communication with a stomach, the distal end of the elongated member having a plurality of tissue grippers that together grip both esophageal tissue oral of a Z line and stomach tissue aboral of the Z line;
a tissue shaper carried on the distal end of and hingedly coupled to the elongated member to cause stomach tissue to be shaped to be received between the elongated member and the tissue shaper when the tissue shaper hingedly pivots into engagement with the elongated member; and
a tissue fastener that maintains the shaped stomach tissue.

18. The assembly of claim 17, further comprising a window between an adjacent pair of the tissue grippers of the elongated member to permit visualization of the Z line.

19. The assembly of claim 18 wherein the tissue fastener is aboral of the window.

20. The assembly of claim 17, wherein at least one of the tissue grippers of the elongated member is a noninvasive gripper.

21. The assembly of claim 20, wherein all of the tissue grippers of the elongated member are noninvasive grippers.

22. The assembly of claim 17, wherein at least one of the tissue grippers of the elongated member is a vacuum gripper.

23. The assembly of claim 22, wherein all of the tissue grippers of the elongated member are vacuum grippers.

24. The assembly of claim 17, wherein the elongated member is arranged to axially translate after the tissue grippers grip esophageal and stomach tissue to cause the Z line to be aboral of an associated diaphragm.

25. A method of restoring a gastroesophageal flap valve associated with an esophagus, a stomach and a Z line that transitions esophageal tissue and stomach tissue, the method comprising the steps of:
concurrently gripping esophageal tissue oral of the Z line and stomach tissue aboral of the Z line;
axially translating the gripped tissue until the Z line is within an associated diaphragm; and
manipulating stomach tissue from within the stomach to restore the gastroesophageal flap valve.

26. The method of claim 25, wherein the concurrently gripping step includes substantially simultaneously gripping the esophageal tissue oral of the Z line and stomach tissue aboral of the Z line.

27. The method of claim 25, wherein the concurrently gripping step includes non-invasively gripping the esophageal tissue oral of the Z line and stomach tissue aboral of the Z line.

28. The method of claim 25, wherein the concurrently gripping step includes vacuum gripping the esophageal tissue oral of the Z line and stomach tissue aboral of the Z line.

29. The method of claim 25, wherein the manipulating step includes folding stomach tissue into a gastroesophageal flap valve shape.

30. The method of claim 25, further comprising the step of fastening the manipulated tissue to maintain the restored gastroesophageal flap valve.

31. The method of claim 25, further comprising first gripping the esophagus oral of the Z line, translating the esophagus in an oral direction, and while maintaining a grip on the esophagus oral of the Z line, gripping the stomach tissue aboral of the Z line to concurrently grip esophageal tissue oral of the Z line and stomach tissue aboral of the Z line.

32. The method of claim 31, wherein the gripping steps include non-invasively gripping the esophageal tissue oral of the Z line and stomach tissue aboral of the Z line.

33. The method of claim 31, wherein the gripping steps include vacuum gripping the esophageal tissue oral of the Z line and stomach tissue aboral of the Z line.

34. The method of claim 31, wherein the manipulating step includes folding stomach tissue into a gastroesophageal flap valve shape.

35. The method of claim 31, further comprising the step of fastening the manipulated tissue to maintain the restored gastroesophageal flap valve.
